(19) United States
(12) Patent Application Publication
(10) Pub. No.: US 2025/0120960 A1
(43) Pub. Date: Apr. 17, 2025

(54) Compositions and Methods for Treatment of Inflammatory Bowel Disease

(71) Applicant: Nocion Therapeutics, Inc., Watertown, MA (US)

(72) Inventors: Bridget McCarthy COLE, Quincy, MA (US); James Lamond ELLIS, Somerville, MA (US)

(21) Appl. No.: 18/925,166

(22) Filed: Oct. 24, 2024

Related U.S. Application Data
(63) Continuation of application No. PCT/US2023/020681, filed on May 2, 2023.
(60) Provisional application No. 63/337,833, filed on May 3, 2022.

(51) Int. Cl. A61K 31/452; A61K 9/00; A61P 1/04

(52) U.S. Cl. CPC A61K 31/452; A61K 9/0053; A61P 1/04

(57) ABSTRACT

The invention relates to the unexpected discovery that the oral administration of charged sodium channel blockers, also called “Nocions,” alleviates inflammation in intestinal inflammatory diseases, such as IBD, ulcerative colitis and/or Crohn's Disease, without substantial systemic exposure, as evidenced by low plasma concentrations.

### RELATED APPLICATIONS

This application is a continuation of International Application No. PCT/US23/20681, which designated the United States and was filed on May 2, 2023, published in English, which claims the benefit of U.S. Provisional Application No. 63/337,833, filed on May 3, 2022. The entire teachings of the above applications are incorporated herein by reference.

### FIELD OF THE APPLICATION

The present invention relates to compositions and methods for treatment of inflammatory bowel disease.

### BACKGROUND

Inflammatory bowel disease (IBD) includes two specific forms of chronic inflammation in the intestinal tract, ulcerative colitis (UC) and Crohn's disease (CD). While clinically distinct, both disorders have features in common, including prominent symptoms of abdominal pain, diarrhea and intestinal dysfunction, accompanied by weakness, fatigue, and weight loss, all of which profoundly interfere with quality of life. In addition to these symptoms, IBD is characterized by clinical signs such as endoscopically visible mucosal ulcerations and swelling, tissue friability, bowel strictures, fistulae, obstructions and abdominal masses.

Treatments for UC and CD have focused on the role of inflammation in their etiologies. For example, tofacitinib and other similar agents act as immune modulators by affecting pathways such as the intracellular JAK-STAT signaling pathway. However, these agents have systemic distribution and affect any cells that have their intended immune-active target, even when administered orally and can have diffuse systemic effects, including the risk of serious infections, venous thrombosis and thromboembolism, and a spectrum of less severe adverse effects. Therefore, a need exists to provide improved therapies with fewer risks.

### SUMMARY

The invention relates to the unexpected discovery that the oral administration of charged sodium channel blockers, also called “Nocions,” alleviates inflammation in intestinal inflammatory diseases, such as IBD, ulcerative colitis and/or Crohn's Disease, without substantial systemic exposure, as evidenced by low plasma concentrations.

### DETAILED DESCRIPTION

Compounds that can be used in the compositions, formulations, and methods of the present invention include charged sodium channel blockers, also called Nocions. One or more Nocions can be formulated in pharmaceutically acceptable compositions or formulations, preferably formulated for oral delivery, such as intestinal delivery. Preferred compositions include sustained release compositions. Preferred Nocions are described in PCT/US2020/22076, filed on Mar. 11, 2020, by Cole et al. (Attorney Docket No. 4270.3001WO); PCT/US2020/21985, filed on Mar. 11, 2020, by Cole et al. (Attorney Docket No. 4270.3002WO); PCT/US2020/59076, filed on Nov. 5, 2020, by Cole et al. (Attorney Docket No. 4270.3003WO); PCT/US2020/59097, filed on Nov. 5, 2020, by Cole et al. (Attorney Docket No. 4270.3004WO); PCT/US2020/22097, filed on Mar. 11, 2020, by Cole et al. (Attorney Docket No. 4270.3005WO); PCT/US2020/21978, filed on Mar. 11, 2020, by Cole et al. (Attorney Docket No. 4270.3006WO); PCT/US2020/22127, filed on Mar. 11, 2020, by Cole et al. (Attorney Docket No. 4270.3007WO); PCT/US2020/22142, filed on Mar. 11, 2020, by Cole et al. (Attorney Docket No. 4270.3008WO); and PCT/US2021/21697, filed on Mar. 10, 2021, by Cole et al. (Attorney Docket No. 4270.3014WO), each of which is incorporated herein in its entirety.

The present invention provides compounds represented by Formula (I):

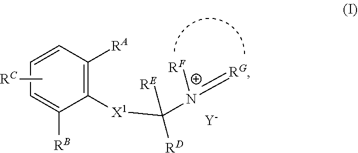

wherein
Y⁻ is a pharmaceutically acceptable anion;
R^{F} and R^{G} together with N⁺ form an optionally substituted heteroaryl ring having one or more heteroatoms or an optionally substituted bicyclic heteroaryl ring having one or more heteroatoms;
R^{A}, R^{B}, and R^{C} are each independently selected from H, D, halogen, substituted or unsubstituted alkyl, substituted or unsubstituted alkenyl, substituted or unsubstituted alkynyl, nitrile, OR^{I}, NR^{J}R^{K}, NR^{L}C(O)R^{M}, S(O)R^{N}, SO₂R^{O}, SO₂R^{O}R^{P}, SO₂NR^{Q}R^{R}, SO₃R^{S}, CO₂R^{T}, C(O)R^{U}, and C(O)NR^{V}R^{W}; or R^{B} and vicinal R^{C} together with the carbon atoms to which they are attached form a substituted or unsubstituted 3-7-membered cycloalkyl (a C₃-C₇ cycloalkyl) or a substituted or unsubstituted aryl (for example, a phenyl);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —OC(O)—, —SC(O)—, —NR^{Z}S(O)—, —S(O)NR^{Z}—, —NR^{X}C(O)NR^{Y}—, —C(O)NR^{1A}—, —C(O)O—, —C(O)—, —S(O)—, —S(O)₂—, and —(O)CS—; X¹ can also be —NR^{Z}C(O)CR^{X}R^{Y}—,
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; and each of R^{D} and R^{E} is independently selected from H, D, substituted or unsubstituted alkyl, substituted or unsubstituted alkenyl, substituted or unsubstituted alkynyl, substituted or unsubstituted heteroalkyl, optionally substituted with halogen, cyclic alkyl, aryl, or heteroaryl, and cycloalkyl; or R^{D} and R^{E} together with the carbon to which they are attached to form a substituted or unsubstituted 3-7-membered cycloalkyl (a C₃-C₇ cycloalkyl) or a substituted or unsubstituted heterocyclic or heteroalkyl ring; or R^{D} and R^{Z} together with the carbon and the —N—C(O)— to which they are attached form an optionally substituted 5-8-membered lactam.

In a preferred embodiment, X¹ is —NHC(O)— or —C(O)NH—. In additional preferred embodiments, X¹ is —NHC(O)—.

In some embodiments, each of R^{A} and R^{B} is independently selected from H, D, halogen, substituted or unsubstituted C₁₋₄ (C₁-C₄) alkyl, and NR^{J}R^{K}; and each of R^{J} and R^{K} is independently selected from H and substituted or unsubstituted C₁₋₄alkyl; and/or wherein R^{C} is not H, such as halogen, C₁₋₄ alkyl, and NR^{J}R^{K}.

In a preferred embodiment, each of R^{A} and R^{B} is —CH₃.

In certain other embodiments, R^{D} is C₁₋₄ alkyl optionally substituted with a substituent selected from the group consisting of halogen, oxygen (oxo), C₃₋₈ (C₃-C₈) cycloalkyl, aryl, and heteroaryl, and/or R^{E} is H or C₁₋₄ alkyl optionally substituted with a substituent selected from the group consisting of halogen, oxygen, C₃₋₈ cycloalkyl, aryl, and heteroaryl.

In preferred embodiments, each of R^{D} and R^{E} is independently selected from —H, —CH₃, —CH₂CH₃, and —(CH₂)₂CH₃. In a more preferred embodiment, R^{E} is hydrogen and R^{D} is —H, —CH₃, —CH₂CH₃, or —(CH₂)₂CH₃.

In certain preferred embodiments, R^{D} and R^{E} are both hydrogen. In yet additional preferred embodiments, R^{D} is hydrogen and R^{E} is an alkyl, for example, a C₁-C₆ alkyl or a C₁-C₄alkyl including, but not limited to, methyl, ethyl, propyl and butyl. In certain additional preferred embodiments, R^{D} and R^{E} are taken together with the carbon to which they are attached to form a C₃-C₆ cycloalkyl including, but not limited to, cyclopropyl or cyclobutyl.

The present invention provides compounds represented by Formula (II):

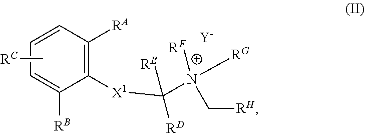

wherein:
Y⁻ is a pharmaceutically acceptable anion;
R^{A}, R^{B}, and R^{C} are each independently selected from H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W}; (preferably H, F, Cl, or CN and more preferably H);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; R^{J} and R^{K} or R^{V} and R^{W} or R^{Q} and R^{R} can also be taken together with the nitrogen to which they are attached to form a substituted or unsubstituted 5, 6, 7, or 8 membered ring;
R^{A}, R^{B}, and/or R^{C} can be taken together with the phenyl ring to which they are attached can form a fused bicyclic or tricyclic ring system, such as naphthyl, dihydroindenyl, tetrahydronaphthyl, quinolinyl, indolyl, and the like;
X¹ is selected from —CR^{X}R^{Y}—, —NR^{Z}C(O)—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; X¹ can also be —NR^{Z}C(O)CR^{X}R^{Y}—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted 3-6-membered cycloalkyl (a C₃-C₆ cycloalkyl), substituted or unsubstituted heterocyclic, or substituted or unsubstituted heteroalkyl ring;
or R^{D} and R^{Z} together with the carbon and the —N—C(O)— to which they are attached form an optionally substituted 5-8-membered lactam;
R^{F} and R^{G} together with N⁺ form an optionally substituted heterocyclic ring having one or more nitrogen atoms; or, each of R^{F} and R^{G} is independently selected from substituted or unsubstituted alkyl, substituted or unsubstituted alkenyl, substituted or unsubstituted alkynyl, substituted or unsubstituted heteroalkyl, and substituted or unsubstituted C₃₋₆ cycloalkyl; and
R^{H} is a substituted or unsubstituted aryl ring, or a substituted or unsubstituted heteroaryl ring.

In another embodiment, R^{H} can be a substituted alkyl. The substituent is preferably an ester group, such as —OC(O)R^{1B} wherein R^{1B} is substituted or unsubstituted alkyl, substituted or unsubstituted alkenyl, substituted or unsubstituted alkynyl, and substituted or unsubstituted heteroalkyl, substituted or unsubstituted aryl, or substituted or unsubstituted heteroaryl. R^{1B} is preferably a substituted or unsubstituted phenyl. R^{H} is preferably —CH₂OC(O)-phenyl.

The present invention provides compounds represented by Formula (III):

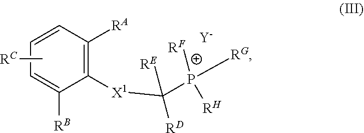

wherein:
Y⁻ is a pharmaceutically acceptable anion;
R^{A}, R^{B}, and R^{C} are each independently selected from H, D, halogen, substituted or unsubstituted alkyl, substituted or unsubstituted alkenyl, substituted or unsubstituted alkynyl, OR^{I}, NR^{J}R^{K}, NR^{L}C(O)R^{M}, CN, S(O)R^{N}, S(O)₂R^{N}, SO₂R^{O}R^{P}, SO₂NR^{Q}R^{R}, SO₃R^{S}, CO₂R^{T}, C(O)R^{U}, and C(O)NR^{V}R^{W};
each of R^{I}, R^{J}, R^{K}, R^{L}, R^{M}, R^{N}, R^{G}, R^{P}, R^{Q}, R^{R}, R^{S}, R^{T}, R^{U}, R^{V}, and R^{W} is independently selected from H, D, halogen, substituted or unsubstituted alkyl, substituted or unsubstituted alkenyl, substituted or unsubstituted alkynyl, and substituted or unsubstituted heteroalkyl;
   X¹ is selected from —CR^{X}R^{Y}—, —NR^{Z}C(O)—, —NR^{Z}C(O)CR^{X}R^{Y}—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; or R^{X}, R^{Y}, R^{Z}, or R^{1A} together with R^{D} or R^{E} and the atoms to which they are attached form an optionally substituted cycloalkyl (such as a C₃-C₆ cycloalkyl) or an optionally substituted heterocyclic ring (such as a 3- to 15-membered heterocyclic ring);
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted cycloalkyl (such as a C₃-C₆ cycloalkyl) or a substituted or unsubstituted heterocyclic (such as a 3- to 15-membered heterocyclic ring);
each of R^{F}, R^{G} and R^{H} is independently selected from H, D, substituted or unsubstituted alkyl, substituted or unsubstituted alkenyl, substituted or unsubstituted alkynyl, substituted or unsubstituted heteroalkyl, substituted or unsubstituted heterocyclyl, substituted or unsubstituted cycloalkyl, substituted or unsubstituted —C₆-C₁₀ aryl, substituted or unsubstituted 5- to 10-membered heteroaryl, substituted or unsubstituted —CH₂—C₅-C₁₀ aryl, and substituted or unsubstituted —CH₂—C₅-C₁₀ heteroaryl; or alternatively, two or three of R^{F}, R^{G} and R^{H} together with the P⁺ to which they are attached form an optionally substituted heterocyclyl (such as a 3- to 15-membered heterocyclic ring) having, zero, one or more heteroatoms in addition to the P⁺, including, but not limited to, an optionally substituted heteroaryl ring;
or two or three of R^{D}, R^{E}, R^{F}, R^{G} and R^{H} together with the P⁺ form an optionally substituted heterocyclic ring having, zero, one or more heteroatoms in addition to the P⁺, including but not limited to, a heteroaryl ring; for example, two of R^{E}, R^{F}, and R^{G} are taken together with the P⁺ to form a heterocyclic ring having, zero, one or more heteroatoms in addition to the P⁺.

The invention further relates to phosphonium derivatives of sodium channel blocker compounds comprising an amine group (for example, a caine compound) wherein the amine of the sodium channel blocker compound is replaced by a phosphonium. Examples of sodium channel blocker compounds include, but are not limited to, lidocaine, bupivacaine, mepivacaine, etidocaine, prilocaine, tocainide, ropivacaine, proparacaine, allocaine, encainide, procainamide, metoclopramide, flecainide, tetracaine, benzocaine, oxybuprocaine, butambine, propoxycaine, dyclonine, pramocaine, chloroprocaine, proparacaine, piperocaine, hexylcaine, naepaine, cyclomethylcaine, and dibucaine, articaine, mexiletine, bupropion, ambroxol, procaine, tolperinone, and substituted derivatives thereof.

The present invention provides compounds represented by Formula (IV):

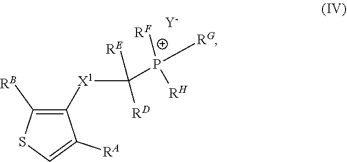

wherein:
Y⁻ is a pharmaceutically acceptable anion;
R^{A} and R^{B} are each independently selected from H, ²H, halogen, substituted or unsubstituted alkyl, substituted or unsubstituted heteroalkyl, substituted or unsubstituted alkenyl, substituted or unsubstituted alkynyl, OR^{I}, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}, C(O)R^{U}, and C(O)NR^{V}R^{W};
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, ²H, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —NR^{Z}C(O)CR^{X}R^{Y}—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—;
each of R^{X}, R^{Y}, R^{Z}, and R^{1A} is independently selected from H, ²H, substituted or unsubstituted alkyl, substituted or unsubstituted alkenyl, substituted or unsubstituted alkynyl, and substituted or unsubstituted heteroalkyl; or R^{X}, R^{Y}, R^{Z}, or R^{1A} together with R^{D} or R^{E} and the atoms to which they are attached form an optionally substituted cycloalkyl (such as a C₃-C₆ cycloalkyl) or an optionally substituted heterocyclic ring (such as a 3- to 15-membered heterocyclic ring);
each of R^{D} and R^{E} is independently selected from H, ²H, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted cycloalkyl (such as a C₃-C₆ cycloalkyl) or a substituted or unsubstituted heterocyclic (such as a 3- to 15-membered heterocyclic ring);
each of R^{F}, R^{G} and R^{H} is independently selected from H, ²H, substituted or unsubstituted alkyl, substituted or unsubstituted alkenyl, substituted or unsubstituted alkynyl, substituted or unsubstituted heteroalkyl, substituted or unsubstituted heterocyclyl, substituted or unsubstituted cycloalkyl, substituted or unsubstituted —C₆-C₁₀ aryl, substituted or unsubstituted 5- to 10-membered heteroaryl, substituted or unsubstituted —CH₂—C₅-C₁₀ aryl, and substituted or unsubstituted —CH₂—C₅-C₁₀ heteroaryl; or alternatively, two or three of R^{F}, R^{G} and R^{H} together with the P⁺ to which they are attached form an optionally substituted heterocyclyl (such as a 3- to 15-membered heterocyclic ring) having, zero, one or more heteroatoms in addition to the P⁺, including, but not limited to, an optionally substituted heteroaryl ring;
or two or three of R^{D}, R^{E}, R^{F}, R^{G} and R^{H} together with the P⁺ to which they are attached form an optionally substituted heterocyclic ring having, zero, one or more heteroatoms in addition to the P⁺, including, but not limited to, a heteroaryl ring; for example, two of R^{E}, R^{F}, and R^{G} are taken together with the P⁺ to form a heterocyclic ring having, zero, one or more heteroatoms in addition to the P⁺.

The present invention provides compounds represented by Formula (V):

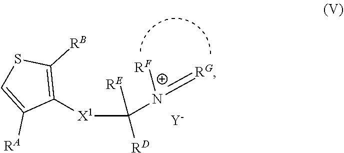

wherein:
Y⁻ is a pharmaceutically acceptable anion;
R^{F} and R^{G} together with the N⁺ to which they are attached form an optionally substituted heteroaryl ring having one or more heteroatoms or an optionally substituted bicyclic heteroaryl ring having one or more heteroatoms;
R^{A} and R^{B} are each independently selected from H, D, halogen, substituted or unsubstituted alkyl, substituted or unsubstituted alkenyl, substituted or unsubstituted alkynyl, nitrile, OR^{I}, NR^{J}R^{K}, NR^{L}C(O)R^{M}, S(O)R^{N}, SO₂R^{O}, SO₂R^{O}R^{P}, SO₂NR^{Q}R^{R}, SO₃R^{S}, CO₂R^{T}, C(O)R^{U}, and C(O)NR^{V}R^{W};
   each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —NR^{Z}C(O)CR^{X}R^{Y}—, —OC(O)—, —SC(O)—, —NR^{Z}S(O)—, —S(O)NR^{Z}—, —NR^{X}C(O)NR^{Y}—, —C(O)NR^{1A}—, —C(O)O—, —C(O)—, —S(O)—, —S(O)₂—, and —(O)CS—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; or can be taken together with any other R^{X}, R^{Y}, R^{Z}, R^{1A}, R^{D} or R^{E} together with any intervening atoms to form a substituted or unsubstituted ring;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, optionally substituted with halogen, cyclic alkyl, aryl, or heteroaryl, and cycloalkyl; or R^{D} and R^{E} together with the carbon to which they are attached to form a substituted or unsubstituted 3-7-membered cycloalkyl (a C₃-C₇ cycloalkyl) or a substituted or unsubstituted heterocyclic or heteroalkyl ring, or R^{D} and R^{Z} together with the carbon and the —N—C(O)— to which they are attached form an optionally substituted 5-8-membered lactam.

In a preferred embodiment, X¹ is —NHC(O)— or —C(O)NH—. In additional preferred embodiments, X¹ is —NHC(O)—.

In some embodiments, R^{A} and R^{B} are independently selected from H, D, halogen, OR^{I}, substituted or unsubstituted C₁-C₄ alkyl, and CO₂R^{T}; wherein R^{I} and R^{T} are independently selected from substituted and unsubstituted C₁-C₄ alkyl.

In a preferred aspect, R^{A} is CH₃ and R^{B} is CO₂R^{T}, wherein R^{T} is selected from H and substituted or unsubstituted C₁₋₄ alkyl. In an additional preferred embodiment, R^{A} is CH₃, and R^{B} is selected from CO₂CH₃ and CO₂CH₂CH₃. In a further preferred embodiment, R^{A} is methyl and R^{B} is C(O)OCH₃. In yet another preferred aspect, R^{A} is methyl and R^{B} is C(O)OCH₂CH₃. In yet another preferred aspect, R^{A} is methyl and R^{B} is methyl.

In certain other embodiments, R^{D} is C₁₋₄ alkyl optionally substituted with a substituent selected from the group consisting of halogen, oxygen (oxo), C₃₋₈ (C₃-C₈) cycloalkyl, aryl, and heteroaryl, and/or R^{E} is H or C₁₋₄ alkyl optionally substituted with a substituent selected from the group consisting of halogen, oxygen, C₃₋₈ cycloalkyl, aryl, and heteroaryl.

In preferred embodiments, each of R^{D} and R^{E} is independently selected from —H, —CH₃, —CH₂CH₃, and —(CH₂)₂CH₃. In a more preferred embodiment, R^{E} is hydrogen and R^{D} is —H, —CH₃, —CH₂CH₃, or —(CH₂)₂CH₃.

In certain preferred embodiments, R^{D} is selected from hydrogen and ethyl and R^{E} is hydrogen. In yet additional preferred embodiments, R^{D} is selected from hydrogen and ethyl and R^{E} is an alkyl, for example, a C₁-C₆ alkyl or a C₁-C₄ alkyl including, but not limited to, methyl, ethyl, propyl and butyl. In further aspects, R^{D} is hydrogen and R^{E} is hydrogen. In yet additional preferred embodiments, R^{D} is ethyl and R^{E} is hydrogen. In certain additional preferred embodiments, R^{D} and R^{E} are taken together with the carbon to which they are attached to form a C₃-C₆ cycloalkyl including, but not limited to, cyclopropyl or cyclobutyl.

In some embodiments Y⁻ includes, but is not limited to, a halide ion, a substituted or unsubstituted alkylsulfonate, a substituted or unsubstituted arylsulfonate, an aliphatic carboxylate, a substituted aliphatic carboxylate, an aryl carboxylate, a substituted aryl carboxylate, a heterocyclyl carboxylate or a substituted heterocyclyl carboxylate.

In additional aspects, Y⁻ is a halide ion. In one embodiment, Y⁻ is the halide ion selected from bromide, chloride, and iodide.

The present invention provides compounds represented by Formula (VI):

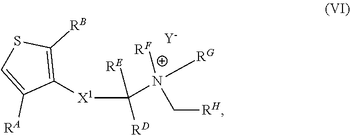

wherein:
Y⁻ is a pharmaceutically acceptable anion;
R^{A} and R^{B} are each independently selected from H, D, halogen, substituted or unsubstituted alkyl, substituted or unsubstituted alkenyl, substituted or unsubstituted alkynyl, OR^{I}, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}, C(O)R^{U}, and C(O)NR^{V}R^{W};
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; X¹ can also be —NR^{Z}C(O)CR^{X}R^{Y}—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; or can be taken together with any other R^{X}, R^{Y}, R^{Z}, R^{1A}, R^{D} or R^{E} together with any intervening atoms to form a substituted or unsubstituted ring;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted 3-6-membered cycloalkyl (a C₃-C₆ cycloalkyl), substituted or unsubstituted heterocyclic, or substituted or unsubstituted heteroalkyl ring; or R^{D} and R^{Z} together with the carbon and the —N—C(O)— to which they are attached form an optionally substituted 5-8-membered lactam;
R^{F} and R^{G} together with the N⁺ to which they are attached form an optionally substituted heterocyclic ring having one or more nitrogen atoms; or, each of R^{F} and R^{G} is independently selected from substituted or unsubstituted alkyl, substituted or unsubstituted alkenyl, substituted or unsubstituted alkynyl, substituted or unsubstituted heteroalkyl, and substituted or unsubstituted C₃₋₆ cycloalkyl; and
R^{H} is a substituted or unsubstituted aryl ring, or a substituted or unsubstituted heteroaryl ring.

In another embodiment, R^{H} can be a substituted alkyl. The substituent is preferably an ester group, such as —OC(O)R^{1B} wherein R^{1B} is substituted or unsubstituted alkyl, substituted or unsubstituted alkenyl, substituted or unsubstituted alkynyl, and substituted or unsubstituted heteroalkyl; substituted or unsubstituted aryl or substituted or unsubstituted heteroaryl. R^{1B} is preferably a substituted or unsubstituted phenyl. R^{H} is preferably —CH₂OC(O)-phenyl.

The present invention provides compounds represented by Formula (VII):

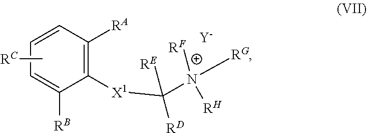

wherein:
Y⁻ is a pharmaceutically acceptable anion;
R^{A} is CO₂R^{T};
R^{T} is substituted or unsubstituted alkyl (preferably methyl or ethyl);
R^{B} is H, D, halogen, or substituted or unsubstituted alkyl; preferably methyl or ethyl;
R^{C} is selected from as H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W}, (preferably H, F, Cl, or CN and more preferably H);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; R^{J} and R^{K} or R^{V} and R^{W} or R^{Q} and R^{R} can also be taken together with the nitrogen to which they are attached to form a substituted or unsubstituted 5, 6, 7, or 8 membered ring;
X¹ can be selected from —CR^{X}R^{Y}—, —NR^{Z}C(O)—, —NR^{Z}C(O)CR^{X}R^{Y}—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; and
each of R^{X}, R^{Y}, R^{Z}, and R^{1A} is independently selected from H, D, substituted or unsubstituted alkyl, substituted or unsubstituted alkenyl, and substituted or unsubstituted alkynyl. In a preferred embodiment, X¹ is —NR^{Z}C(O)—. In additional preferred embodiments, R^{Z} is hydrogen. In yet an additional preferred embodiment, X¹ is —NR^{Z}C(O)— and R^{Z} is hydrogen. In certain preferred embodiments, X¹ is —NHC(O)—;
R^{Z} is H, D, substituted or unsubstituted alkyl, substituted or unsubstituted alkenyl, substituted or unsubstituted alkynyl, and substituted or unsubstituted heteroalkyl (preferably H);
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl;
or R^{D} and R^{E} together with the carbon to which they are attached form a substituted or unsubstituted cycloalkyl (such as a C₃-C₆ cycloalkyl) or a substituted or unsubstituted heterocyclic (such as a 3- to 15-membered heterocyclic ring);
or R^{D} and R^{Z} together with the carbon and the —NC(O)— to which they are attached form a substituted or unsubstituted lactam;
each of R^{F}, R^{G} and R^{H} is independently selected from absent, H, D, substituted or unsubstituted alkyl, substituted or unsubstituted alkenyl, substituted or unsubstituted alkynyl, substituted or unsubstituted heteroalkyl, substituted or unsubstituted heterocyclyl, substituted or unsubstituted cycloalkyl, substituted or unsubstituted —C₆-C₁₀ aryl, substituted or unsubstituted 5- to 10-membered heteroaryl, substituted or unsubstituted —CH₂—C₅-C₁₀ aryl, and substituted or unsubstituted —CH₂—C₅-C₁₀ heteroaryl; or alternatively, two or three of R^{F}, R^{G} and R^{H} together with the N⁺ to which they are attached form an optionally substituted heterocyclyl (such as a 3- to 15-membered heterocyclic ring) having, zero, one or more heteroatoms in addition to the N⁺, including, but not limited to, an optionally substituted heteroaryl ring;
or two or three of R^{D}, R^{E}, R^{F}, R^{G} and R^{H} together with the N⁺ form an optionally substituted heterocyclic ring having, zero, one or more heteroatoms in addition to the N⁺, including but not limited to, a heteroaryl ring; for example, two of R^{E}, R^{F}, and R^{G} are taken together with the N⁺ to form a heterocyclic ring having, zero, one or more heteroatoms in addition to the N⁺.

The invention further relates to derivatives of sodium channel blocker compounds comprising dimethyl anilide (for example, a caine compound) wherein the ortho methyl of the sodium channel blocker compound is replaced by an ester. Examples of sodium channel blocker compounds include, but are not limited to, lidocaine, bupivacaine, mepivacaine, etidocaine, prilocaine, tocainide, ropivacaine, proparacaine, allocaine, encainide, procainamide, metoclopramide, flecainide, tetracaine, benzocaine, oxybuprocaine, butambine, propoxycaine, dyclonine, pramocaine, chloroprocaine, proparacaine, piperocaine, hexylcaine, naepaine, cyclomethylcaine, and dibucaine, articaine, mexiletine, bupropion, ambroxol, procaine, tolperinone, and substituted derivatives thereof.

The present invention provides compounds represented by Formula (VIII):

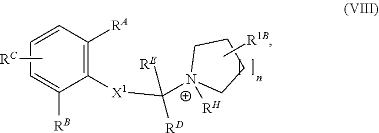

wherein:
Y⁻ is a pharmaceutically acceptable anion;
n is 1, 2, 3, or 4;
R^{A}, R^{B}, and R^{C} are each independently selected from H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W}, (preferably R^{A} and R^{B} are methyl and R^{C} is H, F, Cl, or CN and more preferably H);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; R^{J} and R^{K} or R^{V} and R^{W} or R^{Q} and R^{R} can also be taken together with the nitrogen to which they are attached to form a substituted or unsubstituted 5, 6, 7, or 8 membered ring;
R^{A}, R^{B}, and/or R^{C} taken together with the phenyl ring to which they are attached can form a fused bicyclic or tricyclic ring system, such as naphthyl, dihydroindenyl, tetrahydronaphthyl, quinolinyl, indolyl, and the like;
X¹ is selected from —CR^{X}R^{Y}—, —NR^{Z}C(O)—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; X¹ can also be —NR^{Z}C(O)CR^{X}R^{Y}—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted 3-6-membered cycloalkyl (a C₃-C₆ cycloalkyl), substituted or unsubstituted heterocyclic, or substituted or unsubstituted heteroalkyl ring;
or R^{D} and R^{Z} together with the carbon and the —N—C(O)— to which they are attached form an optionally substituted 5-8-membered lactam;
R^{H} is a substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; in certain aspects, R^{H} can be a substituted or unsubstituted aralkyl or heteroarylalkyl; in additional aspects, R^{H} can be substituted or unsubstituted alkylcarboxylate, substituted or unsubstituted alkylcarboxamide; and
R^{1B} is a substituted or unsubstituted aryl or substituted or unsubstituted heteroaryl, preferably substituted or unsubstituted phenyl.

The present invention provides compounds represented by Formula (IX):

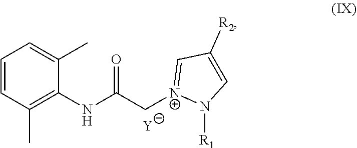

wherein:
Y⁻ is a pharmaceutically acceptable anion;
R¹ is selected from the group consisting of methyl, ethyl, propyl (including n-propyl and isopropyl), butyl (including n-butyl, iso-butyl, tert-butyl, and sec-butyl), cyclohexyl, phenyl, and CH₂C(O)NHR₃;
R² is hydrogen or methyl; and
R³ is hydrogen, methyl, or ethyl.

In some embodiments Y⁻ is a halide ion, a sulfonate (including, for example, a substituted or unsubstituted alkylsulfonate and a substituted or unsubstituted arylsulfonate), or a carboxylate (including, for example, a substituted or unsubstituted alkyl or aliphatic carboxylate, a substituted or unsubstituted aryl carboxylate, or a substituted or unsubstituted heterocyclyl carboxylate).

In additional aspects, Y⁻ is a halide ion. In one embodiment, Y⁻ is the halide ion selected from bromide, chloride, and iodide.

The present invention provides compounds represented by Formula (X):

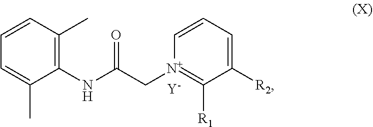

wherein Y⁻ is a pharmaceutically acceptable anion;
one of R¹ and R² is hydrogen, and the other of R¹ and R² is selected from the group consisting of methyl, ethyl, unsubstituted phenyl, and C(O)OR³; and
R³ is selected from the group consisting of hydrogen, methyl, and ethyl.

In some embodiments Y⁻ is a halide ion, a sulfonate (including, for example, a substituted or unsubstituted alkylsulfonate and a substituted or unsubstituted arylsulfonate), carboxylates (including, for example, a substituted or unsubstituted alkyl or aliphatic carboxylate, a substituted or unsubstituted aryl carboxylate, or a substituted or unsubstituted heterocyclyl carboxylate).

In additional aspects, Y⁻ is a halide ion. In one embodiment, Y⁻ is the halide ion selected from bromide, chloride, and iodide.

The present invention provides compounds represented by Formula (XI):

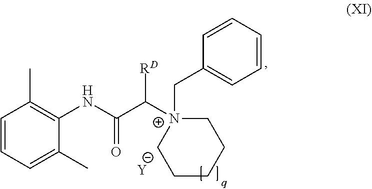

wherein:
Y⁻ is a pharmaceutically acceptable anion;
q is 0, 1, 2, or 3; and
R^{D} is hydrogen, methyl, or ethyl.

The present invention provides compounds represented by Formula (XII):

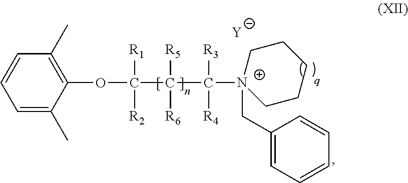

wherein:
Y⁻ is a pharmaceutically acceptable anion;
wherein R₁, R₂, R₃, R₄, R₅, and R₆ are independently selected from hydrogen, C1-C4 alkyl, C1-C4 heteroalkyl, aryl or heteroaryl
n is 0, 1, 2, 3, 4 and 5;
q is 0, 1, 2, 3, 4 or 5.

The present invention provides compounds represented by Formula (XIII):

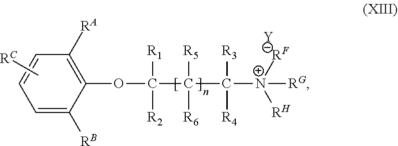

wherein:
Y⁻ is a pharmaceutically acceptable anion;
R^{A}, R^{B}, and R^{C} are each independently selected from H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, CF₃, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W};
each of R^{I}, R^{J}, R^{K}, R^{L}, R^{M}, R^{N}, R^{G}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; or R^{J} and R^{K} or R^{V} and R^{W} or R^{Q} and R^{R} can also be taken together with the nitrogen to which they are attached to form a substituted or unsubstituted 5, 6, 7, or 8 membered ring;
R^{A}, R^{B}, and/or R^{C} can be taken together with the phenyl ring to which they are attached can form a fused bicyclic or tricyclic ring system, such as naphthyl, dihydroindenyl, tetrahydronaphthyl, quinolinyl, indolyl, and the like;
R₁, R₂, R₃, R₄, R₅, and R₆ are independently selected from hydrogen, C₁-C₄ alkyl, C₁-C₄ heteroalkyl, cycloalkyl, aryl or heteroaryl, preferably hydrogen, methyl or ethyl;
n is 0, 1, 2, 3, 4 and 5;
or R₁, R₂, R₃, R₄, R₅, and/or R₆ together with the carbon(s) to which they are attached form a substituted or unsubstituted cycloalkyl (such as a C₃-C₆ cycloalkyl) or a substituted or unsubstituted heterocyclic (such as a 3- to 15-membered heterocyclic ring);
R^{F} and R^{G} together with the N⁺ form an optionally substituted heterocyclic ring having, zero, one or more heteroatoms in addition to the N⁺, including but not limited to, a heteroaryl ring; for example, two of R^{E}, R^{F}, and R^{G} are taken together with the N⁺ to form a heterocyclic ring having, zero, one or more heteroatoms in addition to the N⁺;
R^{H} is selected from substituted or unsubstituted alkyl, substituted or unsubstituted alkenyl, substituted or unsubstituted alkynyl, such as —CH₂-cycloalkyl, —C₂H₄-cycloalkyl, substituted or unsubstituted —CH₂—C₅-C₁₀ aryl, substituted or unsubstituted —C₂H₄—C₅-C₁₀ aryl, substituted or unsubstituted —CH₂—C₅-C₁₀ heteroaryl, substituted or unsubstituted —C₂H₄—C₅-C₁₀ heteroaryl, —CH₂ OC(O)R^{T}, —CH₂ CO₂R^{T}, —CH₂C(O)NR^{V}R^{W}, —C₂H₄OCOR^{T}, —C₂H₄OR^{I} or
R^{F}, R^{G} and R^{H} together with the N⁺ form substituted or unsubstituted heteroaryl ring (such as pyridinyl or phenyl-pyridinyl) or bridged heterocyclic ring.

Charged sodium channel blockers are also described in WO2017/024037 by Bean et al., WO2011/006073 by Woolf et al., WO2008/063603 by Bean et al., and U.S. Pat. No. 8,865,741 by Thompson et al., incorporated herein by reference in their entirety.

Heretofore, a limitation in administering molecules that block sodium channels as treatment agents is that the vast majority of such externally-applied molecules are hydrophobic and can pass readily through membranes. Because of this, they will enter all cells and thus have no selectivity for affecting only nociceptors. This dilutes their therapeutic potential, because the agents are not restricted to acting only on the nociceptors. Moreover, this behavior poses a risk for systemically-mediated side effects, because the molecules reach and affect cells throughout the body. Under certain circumstances, these large pore cationic channels (LPCC) can be activated in the absence of exogenous agonists/ligands by exposure to endogenous inflammatory activators, such as are generated by tissue damage, infection, autoimmunity, atopy, ischemia, hypoxia, cellular stress, immune cell activation, immune mediator production, and oxidative stress. These endogenous inflammation-related substances, including without limitation prostaglandins, nitric oxide (NO), peroxide (H₂O₂), cysteine-reactive inflammatory mediators like 4-hydroxynonenal, endogenous alkenyl aldehydes, endocannabinoids, and immune mediators (e.g., interleukin 1 (IL-1), nerve growth factor (NGF), and bradykinin, can activate the LPCCs expressed on nociceptors and open them, thus allowing charged sodium channel blocking compounds that are unable to pass through cell membranes to enter through the LPCCs into the nociceptor to inhibit the activity of the voltage gated sodium channels thus preventing action potential initiation.

We have identified compounds that are capable of passing through LPCCs that are selectively expressed on visceral nociceptors. Since inflammation opens the LPCCs, the ion channel blockers of the invention can enter these channels on the nociceptors and block the voltage gated sodium channels. However, because the ion channel blocking compounds of the present invention are positively charged, they are not membrane-permeable and thus cannot enter cells in the absence of open LPCCs. Thus, these compounds cannot pass through normal cells.

In addition, the compounds do not enter the systemic circulation upon oral administration. Therefore, ion channel blockers of the invention can selectively target activated visceral nociceptors in the intestinal lumen without affecting or passing through other types of cells. The ability to block the activity of intestinal visceral nociceptors allows these compounds to effectively treat pain mediated by the visceral nociceptors and further to effectively treat manifestations of neurogenic inflammation mediated by intestinal visceral nociceptors, thereby effectively treating one or more symptoms, signs, or sequelae of IBD. Accordingly, the invention provides methods for treating intestinal inflammation, such as IBD, UC or CD, by administering to the patient an effective amount of a composition comprising a Nocion, such as a compound having Formulae (I)—(XIII), wherein the compound inhibits one or more voltage-gated ion channels present in visceral nociceptors, when those channels have been activated by pre-existing inflammation such as is produced in IBD. Preferably, the administration of the composition is an oral administration, which allows the active ingredient to access the affected areas directly as they face the gut lumen.

In embodiments, the LPCC is a transient receptor potential ion channel (TRP channel). In embodiments, the channel is TRPA1, TRPV1-4, TRPM8, ASIC or P2X. In particular embodiments, the compound is capable of entering visceral nociceptors through the TRPA1, TRPV1-4, TRPM8, ASIC or P2X receptor/channel when the receptor/channel is activated, for example by a localized inflammatory process. In embodiments, the methods, compositions, and formulations of the invention treat both nociceptive pain mediated by the visceral nociceptors and neurogenic inflammation mediated by the visceral nociceptors.

Each embodiment described herein can be taken in combination with one, any or all other embodiments, as though presented herein in every permutation. Compositions of the invention can comprise racemic mixtures, pure enantiomers, or an excess of one enantiomer over the other. For example, a composition can comprise an enantiomeric excess of at least 5, 10, 20, 30, 40, 50, 60, 70, 80 or 90%. In one embodiment, the enantiomeric excess is at least 95%. The compounds of the invention include all enantiomers which may be defined, in terms of absolute stereochemistry, as (R)- or (S)-, as well as their racemic and optically pure forms, and is not limited to those described herein in any of their pharmaceutically acceptable forms, including enantiomers, salts, solvates, polymorphs, solvatomorphs, hydrates, anhydrous and other crystalline forms and combinations thereof. Likewise, all tautomeric forms are intended to be included. Preferably, a pharmaceutical composition comprises a compound of the invention as an R enantiomer in substantially pure form; or, a pharmaceutical composition comprises a compound of the invention as an S enantiomer in substantially pure form; or, a pharmaceutical composition comprises a compound of the invention as enantiomeric mixtures which contain an excess of the R enantiomer or an excess of the S enantiomer. It is particularly preferred that the pharmaceutical composition contains a compound of the invention which is a substantially pure optical isomer. For the avoidance of doubt, a compound of the invention can, if desired, be used in the form of solvates.

### Section 1: Definitions

As used herein, the words “a” and “an” are meant to include one or more unless otherwise specified.

As used herein, the term “affected area” refers to those anatomic sites affected by intestinal inflammation, such as IBD, UC or CD, which can include without limitation, areas of mucosal edema, atrophy, ulcers, erosions, breaks, craters, aphthoid lesions, and similar abnormalities and areas of tissue damage.

As used herein, the term “active ingredient” refers to those compositions disclosed herein having biological activity for treating intestinal inflammation, such as IBD, UC or CD. The invention features the inclusion of such active ingredients in formulations for oral administration. As used herein, the terms “treatment,” “treating,” “therapy,” “therapeutic” and the like refer to interventions that are intended to cure, heal, alleviate, improve, remedy, or otherwise beneficially affect a disorder or a condition that impacts the well-being of a patient. A disorder is a condition that alters the homeostatic well-being of a patient, including but not limited to acute or chronic diseases, or pathological conditions that predispose the mammal to an acute or chronic disease. UC and CD, both categorized as IBD, are disorders that are amenable to treatment with the compositions and formulations of the present invention.

By “inflammation” is meant a localized reaction to injury, tissue damage, or other stimuli such those caused by the immune system (immune-mediated inflammation) and/or by the nervous system (neurogenic inflammation), typically resulting in symptoms, including redness, heat, swelling, pain, tissue damage, and/or loss of function. By “neurogenic inflammation” is meant inflammation mediated or contributed to by neurons (e.g. nociceptors) or other components of the central or peripheral nervous system. The term “inflammation” as used herein can refer to acute or chronic inflammation.

By “patient” is meant an animal. In one embodiment, the patient is a human. Other animals that can be treated using the methods, compositions, and kits of the invention include but are not limited to non-human primates (e.g., monkeys, gorillas, chimpanzees), domesticated animals (e.g., horses, pigs, goats, rabbits, sheep, cattle, llamas), and companion animals (e.g., guinea pigs, rats, mice, lizards, snakes, dogs, cats, fish, hamsters, and birds).

Compounds useful in the invention include, but are not limited to, those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. The term “pharmaceutically acceptable” represents those substances which are, within the scope of sound medical judgment, suitable for use in contact with the tissues of humans and lower animals without undue toxicity, irritation, allergic response and the like, and are commensurate with a reasonable benefit/risk ratio. Compounds useful in the invention include pharmaceutically acceptable anions thereof, and charged moieties thereof. The term “pharmaceutically acceptable anion” as used herein, refers to the conjugate base of a pharmaceutically acceptable acid. Such acids are described in Stahl, P. H. and Wermuth, C. G. (eds.), Handbook of Pharmaceutical Salts: Properties, Selection and Use, Wiley VCH (2008). Pharmaceutically acceptable acids include, but are not limited to, acetic acid, dichloroacetic acid, adipic acid, alginic acid, L-ascorbic acid, L-aspartic acid, benzenesulfonic acid, 4-acetamidobenzoic acid, benzoic acid, p-bromophenylsulfonic acid, (+)-camphoric acid, (+)-camphor-10-sulfonic acid, capric acid, caproic acid, caprylic acid, carbonic acid, cinnamic acid, cyclamic acid, dodecylsulfuric acid, ethane-1,2-disulfonic acid, ethanesulfonic acid, 2-hydroxyethanesulfonic acid, sulfuric acid, boric acid, citric acid, formic acid, fumaric acid, galactaric acid, gentisic acid, D-glucoheptonic acid, D-gluconic acid, D-glucuronic acid, glutamic acid, glutaric acid, 2-oxoglutaric acid, glycerophosphoric acid, glycolic acid, hippuric acid, hydrochloric acid, hydrobromic acid, hydroiodic acid, isobutyric acid, DL-lactic acid, lactobionic acid, lauric acid, maleic acid, (−)-L-malic acid, malonic acid, DL-mandelic acid, methanesulfonic acid, naphthalene-1,5-disulfonic acid, naphthalene-2-sulfonic acid, 1-hydroxy-2-naphthoic acid, nicotinic acid, nitric acid, oleic acid, orotic acid, oxalic acid, palmitic acid, pamoic acid, phosphoric acid, propionic acid, (−)-L-pyroglutamic acid, salicyclic acid, 4-aminosalicyclic acid, sebacic acid, stearic acid, succinic acid, (+)-L-tartaric acid, thiocyanic acid, p-toluenesulfonic acid, and undecylenic acid. Pharmaceutically acceptable anions include the conjugate base of any the acids set forth above. By “charged moiety” is meant a moiety which gains a proton at physiological pH thereby becoming positively charged (e.g., ammonium, guanidinium, or amidinium) or a moiety that includes a net formal positive charge without protonation (e.g., quaternary ammonium). The charged moiety is preferably permanently charged. Pharmaceutically acceptable salts can be prepared in situ during the final isolation and purification of the compounds of the invention, or separately by reacting the free base function with a suitable organic acid. Representative acid addition salts include but are not limited to acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

The compounds of the present invention, including salts of the compounds, can exist in unsolvated forms as well as solvated forms, including hydrated forms and unhydrated forms. In general, the solvated forms are equivalent to unsolvated forms and are encompassed within the scope of the present invention. Nonlimiting examples of hydrates include monohydrates, dihydrates, hemihydrates, etc. In certain aspects, the compound is a hemihydrate. Nonlimiting examples of solvates include ethanol solvates, acetone solvates, etc. “Solvates” means solvent addition forms that contain either stoichiometric or nonstoichiometric amounts of solvent. The compounds of the invention may exist in multiple crystalline or amorphous forms. In general, all physical forms are equivalent for uses contemplated by the present invention and are intended to be within the scope of the invention.

By “therapeutically effective amount” or “effective amount” means an amount sufficient to produce a desired result, for example, the reduction or elimination of symptoms, signs, or sequelae associated with or due to IBD, or to decrease the incidence or reduce the severity of other diseases associated with IBD such as colon cancer. As used herein, the term “symptom” refers to those subjectively experienced manifestations of a disease or disorder, while a “sign” is a manifestation of the disease or disorder that is perceived by an external observer, and “sequelae” are long-term consequences of the disease or disorder.

### Section 2: Formulations

a. Formulations Generally

Formulations comprising compounds of the present invention to be used for treating intestinal inflammation, such as IBD, US or CD, can be prepared for oral administration. Dosage forms prepared for oral administration can include liquids or solids. Advantageously, the oral dosage forms will be prepared as sustained release formulations, in order to delay the release of the active ingredient(s) (the Nocion or Nocions) for a time period that is sufficient to allow the dosage form to pass through the intestine via peristalsis until it reaches the local area in need of treatment. As used herein the term “sustained release” refers to any formulation for an oral composition that releases the active ingredient from the oral dosage form at a rate that is slower than immediate release or is controlled to release at a specific location within the intestine. The term “sustained release” can be applied to compositions having a controlled release component or a delayed release component, or both, as will be described in more detail below.

Liquid formulations for oral use can incorporate aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. In keeping with the present invention, Nocions can be dissolved or suspended in liquid vehicles. Liquid oral sustained release systems can be produced, allowing the delivery of the active ingredient intraluminally to one or more affected areas in order to have direct effect thereupon. Techniques for preparing liquid oral sustained release systems are familiar to skilled artisans. As applied to the present invention, liquid sustained release techniques can include, without limitation, the use of controlled release spherical particles containing the Nocions that are suspended in liquid vehicle, including liposomes; the use of ion exchange resins or sparingly soluble salts to produce colloids or suspensions having sustained release properties; and the use of gelling system, such as alginate- or chitosan-based gels, having sustained release properties.

Solid formulations for oral use include tablets containing the Nocion(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, taste masking agents (such as hydroxypropyl methylcellulose, hydroxypropyl cellulose), and the like. One or more compounds of the invention may be mixed together in a tablet, capsule, or other vehicle, or may be partitioned, and may optionally be combined with other biologically active agents that contribute to the treatment of IBD. Formulations for oral use may also be provided as chewable tablets, or as hard gelatin capsules wherein the active ingredient is mixed with an inert solid diluent (e.g. potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus, or spray drying equipment.

b. Exemplary Sustained Release Formulations

Solid oral sustained release systems can be produced that allow the delivery of the active ingredient intraluminally to one or more affected areas in order to have direct therapeutic effect thereupon. Sustained release of the active ingredient from a solid dosage form can be accomplished by any means known in the art, including without limitation the use of matrix dosage forms, multiparticulate dosage forms, reservoirs and pulsatile dosage forms. Such dosage forms can also be prepared for delayed release of the active ingredient. Features characteristic of both delayed release systems and controlled release systems can be incorporated in the same dosage form.

In embodiments, matrix dosage forms can be prepared for controlled release by incorporating the active ingredient(s) in an appropriate matrix. As the matrix is digested or degenerates, it releases the active ingredient over time, producing controlled release. In general, matrix materials can include erodible or non-erodible polymeric matrices: an erodible matrix comprises water-swellable, water-soluble, or water-erodible polymers that contain and support the active ingredient. Then, as the aqueous-swollen matrix passes through the intestine, it gradually degenerates, releasing the active ingredient it supports into the ambient environment. Polymers suitable for use to form such matrices are familiar in the art, including linear, branched, or crosslinked homopolymers or copolymers, for example, naturally occurring polysaccharides such as chitosan, dextran, pullulan and the like; starches such as dextrin and maltodextrin; gums such as xanthan gum, guar gum, carrageenans, gum arabic, and the like; hydrophilic colloids like pectin; alginates; gelatins; collagen; polyvinyl pyrrolidone (PVP), polyvinyl alcohol, polyacrylamide, polyacrylates, glycerol fatty acid esters, and the like; polymers comprising ethacrylic acid or methacrylic acid (EUDRAGIT®), or comprising other acrylic acid derivatives; and cellulosics, such as ethyl cellulose, methylethyl cellulose, carboxymethyl cellulose, hydroxyethyl cellulose, hydroxypropyl cellulose, and the like. Exemplary matrix materials can include substances such as hydrated methylcellulose, carnauba wax and stearyl alcohol, carbopol 934, silicone, glyceryl tristearate, methyl acrylate-methylmethacrylate, polyvinyl chloride, polyethylene, and/or halogenated fluorocarbons.

An erodible matrix system can contain other additives and excipients familiar to practitioners in the pharmaceutical arts, including without limitation, solubility enhancers or retardants, stability enhancers, osmagens, binders, buffers, and the like. In a non-erodible matrix system, the active ingredient is distributed within an inert matrix, and is released by diffusion through the matrix into the ambient environment. Materials used to form an inert matrix include various polymeric materials that are relatively insoluble, including plastics such as copolymers of ethylene and vinyl acetate, methyl acrylate-methyl meth-acrylate copolymers, polyvinyl chloride, and polyethylene; hydrophilic polymers such as cellulose acetate, ethyl cellulose, and crosslinked PVP; waxes, triglycerides and fatty compounds. Further examples, as would be familiar to skilled artisans, can be found in standard references such as Remington: The Science and Practice of Pharmacy, 23rd edition (2020).

Matrices can also be prepared that support a plurality of particles that contain the active ingredient, with the particles comprising a mixture of the active ingredient and a secondary matrix material, wherein the secondary matrix material further modifies the release of the active ingredient. This arrangement produces a multiparticulate dosage form. The secondary matrix material can itself be substantially water soluble or water insoluble, in either case including agents to modify the release of the active ingredient from the secondary matrix.

In embodiments, reservoir systems can be prepared for controlled release of active ingredients such as Nocions. In a reservoir system, a reservoir containing the active ingredient is surrounded by a rate-limiting membrane. The active ingredient is dispensed into the ambient environment by passing through the membrane, using mass transport mechanisms familiar in the art, such as diffusion across the membrane or dissolution into liquid-filled pores in the membrane itself. A reservoir system can be formed with a single large reservoir within the dosage form, for example, within a tablet, or it can involve multiple particles, for example, as a capsule containing a plurality of reservoir particles, each encapsulated by its own membrane. As would be understood by skilled artisans, the membrane can be amorphous or crystalline, and can have any sort of morphology or composition that allows it to provide controlled permeability to the active ingredient, for example, a porous hydrophilic membrane, an interfacially polymerized membrane, a hydrogel membrane, an ionic membrane, and the like. The rate of release of the active ingredient can be engineering various aspects of the reservoir system, such as the composition of and binders used in the active ingredient deposit within the reservoir, the nature and thickness of the membrane, the surface-to-volume ratio of the reservoir elements (whether unicameral or multiparticulate), and the like.

Pulsatile dosage forms suitable for controlled release comprise a tablet core or bead core bearing the active ingredient surrounded by a semipermeable membrane that can be destroyed or disrupted by contact with the environment, leading to a substantially complete and immediate release of the active ingredient when the membrane bursts. Such systems are classified into three categories: time-controlled release, in which the degradation of the external layer takes place as a function of time, for example as the surface erodes or as the internal environment of the system accumulates water and swells; stimulus-induced release, in which the degradation occurs in response to an environmental stimulus, such as pH or exposure to hydrolytic enzymes; and externally induced, with degradation occurring after exposure to external stimuli such as ultrasound or electromagnetic radiation. Technologies are familiar in the art for producing such systems, which can allow control of the timing for release of the Nocions in the small or large intestine so that they are optimally positioned to reach the area(s) in need of treatment.

c. Coatings

Any of the aforesaid methods for sustained release can be moderated or regulated by application of one or more external coating layers. Coatings can be applied in one or more layers to the external surface of the dosage form, and/or they can be applied in one or more layers to the subcomponents within the dosage form such as the active-ingredient-bearing particles that form part of a multiparticulate system.

Coatings are advantageous for preparing delayed release dosage forms as well as controlled release dosage forms, by applying coatings to the entire dosage form or to components thereof that have desirable properties for sustained release. As an example, a tablet can be formed comprising the active ingredient compressed into a core matrix that is coated with a first coating that controls the release of the active ingredient over time during the transit through the intestine, with a second layer covering the entire dosage form that prevents the release of the active ingredient entirely for a certain period of time or until a certain biological environment is reached. For example, a twice-coated tablet formulated to include one or more Nocions can be ingested and pass through the stomach relatively intact if it bears an outer coating selected to resist the acid conditions there, keeping the underlying inner coating and the tablet intact. The dosage form then passes into the duodenum, where the higher pH can disrupt (erode, dissolve, etc.) the acid-resistant outer coating to expose the inner coating. The inner coating can be formulated to produce controlled release of the active ingredient from the underlying tablet. Once the outer coating has been disrupted, the controlled release properties of the inner coating can determine the release profile for the active ingredient. Alternatively, if only an outer coating is provided, it can protect the dosage form sufficiently to delay release of the active ingredient altogether, with subsequent controlled release of the active ingredient being regulated by the properties of the matrix or vehicle within which it is carried.

Suitable materials for the outer delayed release coating are familiar in the art, including those agents generally termed enteric coatings for delayed-release pharmaceutical products. Enteric coatings known in the art are typically resistant to stomach acid and do not dissolve at a pH less than 4; they can therefore protect the underlying tablet from dissolution as it passes through the stomach. Examples of enteric coatings, suitable for use as an outer delayed release layer, include cellulose derivatives such as cellulose acetate phthalate, cellulose acetate trimellitate, hydroxypropyl methyl cellulose acetate succinate, cellulose acetate succinate, carboxy methyl ethyl cellulose, methylcellulose phthalate, hydroxypropyl methylcellulose phthalate, and ethylhydroxy cellulose phthalate; vinyl polymers, such as polyvinyl acetate phthalate, vinyl acetate-maleic anhydride copolymer; polyacrylates; and polymethacrylates such as methyl acrylate-methacrylic acid copolymer, methacrylate-methacrylic acid-octyl acrylate copolymer; and styrene-maleic monoester copolymer, any of which can be used alone or in combination. Certain of these materials are pH sensitive, including as cellulose acetate phthalate, hydroxypropyl methylcellulose phthalate, polyvinyl acetate phthalate, and acrylic copolymers, such as have been commercialized as part of the Eudragit® family (Rohm Pharma), including Eudragit® L 100, Eudragit® S 100, and Eudragit® L30 D 55.

Controlled release coatings can be engineered to provide an appropriate delivery schedule for the active ingredient so that it is allowed to reach the affected area or areas of the intestine. It is understood that the affected areas in the intestine are the treatment targets for the active ingredient. In CD, the affected areas are found in the colon; therefore, the active ingredient needs to reach the colon without being absorbed more proximally if it is to access and treat the lesions through an intraluminal approach. For CD, the lesions can involve the entire intestinal tract, in both the small and the large intestine. Therefore, it is desirable that a controlled release coating protects the active ingredient sufficiently so that it can access the various sites that are affected by the disease, potentially including the entire length of the small bowel and the colon.

In embodiments, the release is directed specifically to the vicinity of one or more affected areas. In other embodiments, the active ingredient can be released proximally and progress within the intestine to treat the lesions that it encounters during transit, provided that significant absorption of the active ingredient does not occur. Controlled release coatings for these purposes include those agents familiar in pharmaceutical formulation and safe for oral administration, including without limitation agents or combinations of agents such as shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols.

### Section 3: Methods of Treatment

Disclosed herein are methods of treatment for intestinal inflammation, such as IBD, UC and CD, comprising the oral administration of a therapeutically effective amount of one or more Nocions, or compounds of the present invention, to a patient in need thereof. Advantageously, the oral administration involves providing a sustained release formulation, which can provide delayed release of the active ingredient, controlled release of the active ingredient, or both. The sustained release formulation can be engineered so that the release is maximized at a selected location in the bowel, based on the patient's clinical needs. For example, a sustained release formulation for an IBD patient with UC can be targeted for maximum release in the colon, with the formulation providing for delayed release until the majority of the small bowel has been traversed or until the colon itself has been reached. A sustained release formulation for an IBD patient with CD can be targeted for release along the length of the small bowel, with a delayed release to ensure passage through the stomach and with a controlled release to allow the active ingredient to access a multitude of affected areas along the length of the small bowel.

Generally, the oral dosage of any of the compounds of the invention administered to a human will depend on the nature of the compound, and can readily be determined by one skilled in the art. Typically, such dosage can be about 0.001 mg to 2000 mg per day, desirably about 1 mg to 1000 mg per day, and more desirably about 5 mg to 500 mg per day. Dosages up to 200 mg per day or more may be necessary. Administration of the compound or compounds can be daily, every other day, or at other intervals. The doses can be provided once, twice, three, or four times daily, or more frequently. Duration of treatments can range from a single day to an entire year or longer, recognizing that IBD is a lifelong illness that can require prolonged or lifelong treatment to prevent symptoms, pathological changes in the intestinal tissues, or sequelae such as colon cancer.

It is understood that IBD itself is considered incurable, with variable symptoms and frequent exacerbations. However, clinical symptoms may not correspond linearly to the anatomic state of the disease or its progress. Therefore objective measures are useful to assess and monitor the activity of the disease. Physicians at present rely on a combination of symptoms, radiological investigation, histological examination of tissue specimens, endoscopy, and laboratory indices to make decisions about treatment. These technologies as currently practiced and as improved in the future can collectively be termed “monitoring” of IBD. Biomarkers allowing monitoring to have been identified in blood, stool, urine, breath, and tissue, and many others are under investigation. The use of monitoring technologies can allow for surveillance of disease activity, evaluating the efficacy of treatments, and predicting the future course of the disease. Monitoring technologies in current practice include, without limitation, detection and measurement of serological antibodies such as autoantibodies and anti-microbial antibodies, detection and measurement of circulating non-coding RNAs, serum metabolic profiling, serum protein profiling, detection and measurement of identified disease markers such as oncostatin M, serum galectins, C-Reactive Protein, fecal calprotectin, pro-inflammatory cytokines, vitamin D levels, and the like.

Dose amounts and schedules can be arranged in response to clinical indications, such as a flare-up or a persistence of symptoms. Doses can also be arranged in light of objective measures of disease persistence, disease progression, or disease regression. In embodiments, a treatment protocol including a prearranged dosage schedule can be adapted to a patient's clinical needs by referring to one or more objective measures such as biomarkers that reflect the state of the disease or the state of inflammation thought to be attributable to the disease. In embodiments, after obtaining baseline objective measurements corresponding to the state of the disease a predefined induction dose is administered for a first period of time, following which a second set of objective measurements is obtained. Based on the second set of objective measurements, a second dose or dosing schedule is determined and administered. In embodiments, the second dose or dosing schedule is less than the induction dose or less frequent than the induction dosing schedule, and in embodiments this second treatment regimen is intended for prolonged administration. In embodiments, a third set of objective measurements is made during the second treatment regimen, following which the dose amount and/or the dosing schedule can be modified. In other embodiments, no induction dose is administered, but objective measurements are made before commencing therapy and at regular intervals during treatment, with dosing being adjusted accordingly.

Treatment of IBD using the methods of the invention can include adjusting the amount of the active ingredient in a single dose, the frequency or schedule of administration, the type of dosage form for delivering the active ingredient, or other parameters that affect the amount of active ingredient that reaches a particular affected area in the intestinal tract within a predetermined time frame (collectively, “modifying” the treatment regimen). Information obtained from monitoring, as described above, can be used to modify the treatment regimen to optimize a course of therapy. In an embodiment, a change in one or more monitoring parameters can be used to increase or decrease the amount of active ingredient administered in a single dose, or to change the frequency of dosing in a single 24 hour period, or to alter the duration of treatment (for example, permitting intermittent courses of treatment of varying duration); other modifications of the treatment regimen based on the monitoring parameters can be envisioned by those of ordinary skill in the art.

### EXAMPLES

### Example 1: Anti-Inflammatory Activity of Nocions in Dextran Sodium (DSS) Model of Ulcerative Colitis

As an exemplary Nocion, a charged sodium channel blocker represented by the following Formula (XIV) (termed “NTX 1170”) was administered in a DSS mouse model of ulcerative colitis, with a comparison of its anti-inflammatory effects with those of tofacitinub and infliximab.

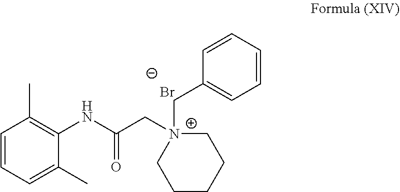 [SMILES: Cc1cccc(C)c1NC(=O)C[N+]1(Cc2ccccc2)CCCCC1 ; [Br-]]

The anti-inflammatory efficacy of Nocions, as exemplified by the compound of Formula XIV (NTX 1170) (100 mg/kg QD PO), was compared with infliximab (5 mg/kg QD IP) and tofacitinib (5 mg/kg BID PO) in mice given 2.5% DSS in drinking water for 6 days. Body weights, stool consistency and the presence of blood in the stool were measured over the 6 days and comprised the Disease Activity Index (DAI). On day 7 the study was terminated, and the colon and plasma were collected for cytokine measurements, histology evaluation and for pharmacokinetic (PK) analysis.

The compound of Formula XIV produced a 60% (P<0.001) improvement in the DAI, compared to a 38% (P<0.05) reduction with tofacitinib and a 14% (NS) reduction with infliximab. Several plasma cytokines (IP-10, MIG, TNF-α, G-CSF and Mip-1β) were upregulated by the DSS treatment and significantly reduced by NTX-1170. Tofacitinib significantly reduced MIG, TNF-α, G-CSF and Mip-1β levels whereas infliximab only reduced TNF-α, G-CSF and Mip-1β levels. Colon KC, IL-15 and RANTES levels were significantly increased by the DSS treatment and significantly reduced by NTX-1170 whereas neither tofacitinib nor infliximab significantly reduced these cytokines. Histopathological analysis of the colon revealed that NTX-1170 significantly reduced the degree of inflammatory infiltrate, the layer to which the infiltration extended and the amount of crypt damage. Tofacitinib only reduced the extent of infiltration whereas infliximab has no significant effect. Analysis of terminal plasma and colon samples showed very low levels of the compound of Formula XIV in the plasma and high levels in the colon with a colon/plasma ratio>3500.

These data indicate that charged sodium channel blocking compounds as exemplified by the compound shown in Formula II inhibit neurogenic inflammation to provide significant efficacy in the DSS model of UC, thus offering treatment options for UC with the potential for greater efficacy than current treatments like infliximab and tofacitinib.

While this invention has been particularly shown and described with references to preferred embodiments thereof, it will be understood by those skilled in the art that various changes in form and details may be made therein without departing from the scope of the invention encompassed by the appended claims. While specific embodiments of the subject invention have been disclosed herein, the above specification is illustrative and not restrictive. Many variations of the invention will become apparent to those of skilled art upon review of this specification; those modifications of the described modes for carrying out the invention that are apparent to those skilled in the fields of medicine, immunology, pharmacology, endocrinology, or related fields are intended to be within the scope of the invention.

Unless otherwise indicated, all numbers expressing reaction conditions, quantities of ingredients, and so forth, as used in this specification and the claims are to be understood as being modified in all instances by the term “about.” Accordingly, unless indicated to the contrary, the numerical parameters set forth herein are approximations that can vary depending upon the desired properties sought to be obtained by the present invention.

1. A method of treating intestinal inflammation comprising oral administration of a pharmaceutical formulation comprising a therapeutically effective amount of a charged sodium channel blocker.

2. The method of claim 1, wherein the intestinal inflammation is selected from the group consisting of inflammatory bowel disease, ulcerative colitis and Crohn's disease.

3. The method of claim 1, wherein the formulation is engineered to release the composition at a selected location of the bowel.

4. The method of claim 1, wherein the formulation is an enteric coated tablet.

5. The method of claim 1, wherein the compound has Formula (I): 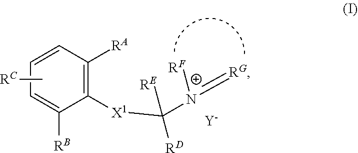
wherein
Y⁻ is a pharmaceutically acceptable anion;
R^{F} and R^{G} together with N⁺ form an optionally substituted heteroaryl ring having one or more heteroatoms or an optionally substituted bicyclic heteroaryl ring having one or more heteroatoms;
R^{A}, R^{B}, and R^{C} are each independently selected from H, D, halogen, substituted or unsubstituted alkyl, substituted or unsubstituted alkenyl, substituted or unsubstituted alkynyl, nitrile, OR^{I}, NR^{J}R^{K}, NR^{L}C(O)R^{M}, S(O)R^{N}, SO₂R^{O}, SO₂R^{O}R^{P}, SO₂NR^{Q}R^{R}, SO₃R^{S}, CO₂R^{T}, C(O)R^{U}, and C(O)NR^{V}R^{W}; or R^{B} and vicinal R^{C} together with the carbon atoms to which they are attached form a substituted or unsubstituted 3-7-membered cycloalkyl (a C₃-C₇ cycloalkyl) or a substituted or unsubstituted aryl (for example, a phenyl);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —OC(O)—, —SC(O)—, —NR^{Z}S(O)—, —S(O)NR^{Z}—, —NR^{X}C(O)NR^{Y}—, —C(O)NR^{1A}—, —C(O)O—, —C(O)—, —S(O)—, —S(O)₂—, and —(O)CS—; X¹ can also be —NR^{Z}C(O)CR^{X}R^{Y}—,
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; and each of R^{D} and R^{E} is independently selected from H, D, substituted or unsubstituted alkyl, substituted or unsubstituted alkenyl, substituted or unsubstituted alkynyl, substituted or unsubstituted heteroalkyl, optionally substituted with halogen, cyclic alkyl, aryl, or heteroaryl, and cycloalkyl; or R^{D} and R^{E} together with the carbon to which they are attached to form a substituted or unsubstituted 3-7-membered cycloalkyl (a C₃-C₇ cycloalkyl) or a substituted or unsubstituted heterocyclic or heteroalkyl ring; or R^{D} and R^{Z} together with the carbon and the —N—C(O)— to which they are attached form an optionally substituted 5-8-membered lactam.

6. The method of claim 1, wherein the compound has Formula (II): 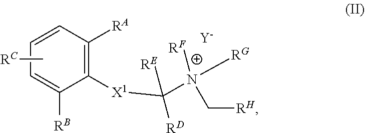
wherein:
Y⁻ is a pharmaceutically acceptable anion;
R^{A}, R^{B}, and R^{C} are each independently selected from H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W}; (preferably H, F, Cl, or CN and more preferably H);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; R^{J} and R^{K} or R^{V} and R^{W} or R^{Q} and R^{R} can also be taken together with the nitrogen to which they are attached to form a substituted or unsubstituted 5, 6, 7, or 8 membered ring;
R^{A}, R^{B}, and/or R^{C} can be taken together with the phenyl ring to which they are attached can form a fused bicyclic or tricyclic ring system, such as naphthyl, dihydroindenyl, tetrahydronaphthyl, quinolinyl, indolyl, and the like;
X¹ is selected from —CR^{X}R^{Y}—, —NR^{Z}C(O)—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; X¹ can also be —NR^{Z}C(O)CR^{X}R^{Y}—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted 3-6-membered cycloalkyl (a C₃-C₆ cycloalkyl), substituted or unsubstituted heterocyclic, or substituted or unsubstituted heteroalkyl ring;
or R^{D} and R^{Z} together with the carbon and the —N—C(O)— to which they are attached form an optionally substituted 5-8-membered lactam;
R^{F} and R^{G} together with N⁺ form an optionally substituted heterocyclic ring having one or more nitrogen atoms; or, each of R^{F} and R^{G} is independently selected from substituted or unsubstituted alkyl, substituted or unsubstituted alkenyl, substituted or unsubstituted alkynyl, substituted or unsubstituted heteroalkyl, and substituted or unsubstituted C₃₋₆ cycloalkyl; and
R^{H} is a substituted or unsubstituted aryl ring, or a substituted or unsubstituted heteroaryl ring.

7. The method of claim 1, wherein the compound has Formula (III): 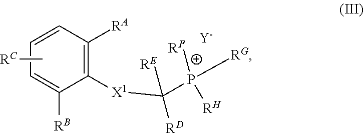
wherein:
Y⁻ is a pharmaceutically acceptable anion;
R^{A}, R^{B}, and R^{C} are each independently selected from H, D, halogen, substituted or unsubstituted alkyl, substituted or unsubstituted alkenyl, substituted or unsubstituted alkynyl, OR^{I}, NR^{J}R^{K}, NR^{L}C(O)R^{M}, CN, S(O)R^{N}, S(O)₂R^{N}, SO₂R^{O}R^{P}, SO₂NR^{Q}R^{R}, SO₃R^{S}, CO₂R^{T}, C(O)R^{U}, and C(O)NR^{V}R^{W};
each of R^{I}, R^{J}, R^{K}, R^{L}, R^{M}, R^{N}, R^{G}, R^{P}, R^{Q}, R^{R}, R^{S}, R^{T}, R^{U}, R^{V}, and R^{W} is independently selected from H, D, halogen, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —NR^{Z}C(O)CR^{X}R^{Y}—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; or R^{X}, R^{Y}, R^{Z}, or R^{1A} together with R^{D} or R^{E} and the atoms to which they are attached form an optionally substituted cycloalkyl (such as a C₃-C₆ cycloalkyl) or an optionally substituted heterocyclic ring (such as a 3- to 15-membered heterocyclic ring);
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted cycloalkyl (such as a C₃-C₆ cycloalkyl) or a substituted or unsubstituted heterocyclic (such as a 3- to 15-membered heterocyclic ring);
each of R^{F}, R^{G} and R^{H} is independently selected from H, D, substituted or unsubstituted alkyl, substituted or unsubstituted alkenyl, substituted or unsubstituted alkynyl, substituted or unsubstituted heteroalkyl, substituted or unsubstituted heterocyclyl, substituted or unsubstituted cycloalkyl, substituted or unsubstituted —C₆-C₁₀ aryl, substituted or unsubstituted 5- to 10-membered heteroaryl, substituted or unsubstituted —CH₂—C₅-C₁₀ aryl, and substituted or unsubstituted —CH₂—C₅-C₁₀ heteroaryl; or alternatively, two or three of R^{F}, R^{G} and R^{H} together with the P⁺ to which they are attached form an optionally substituted heterocyclyl (such as a 3- to 15-membered heterocyclic ring) having, zero, one or more heteroatoms in addition to the P⁺, including, but not limited to, an optionally substituted heteroaryl ring;
or two or three of R^{D}, R^{E}, R^{F}, R^{G} and R^{H} together with the P⁺ form an optionally substituted heterocyclic ring having, zero, one or more heteroatoms in addition to the P⁺, including but not limited to, a heteroaryl ring; for example, two of R^{E}, R^{F}, and R^{G} are taken together with the P⁺ to form a heterocyclic ring having, zero, one or more heteroatoms in addition to the P⁺.

8. The method of claim 1, wherein the compound has Formula (IV): 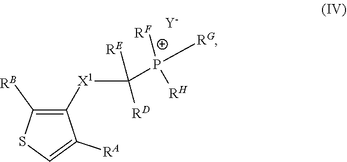
wherein:
Y⁻ is a pharmaceutically acceptable anion;
R^{A} and R^{B} are each independently selected from H, ²H, halogen, substituted or unsubstituted alkyl, substituted or unsubstituted heteroalkyl, substituted or unsubstituted alkenyl, substituted or unsubstituted alkynyl, OR^{I}, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}, C(O)R^{U}, and C(O)NR^{V}R^{W};
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, ²H, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —NR^{Z}C(O)CR^{X}R^{Y}—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—;
each of R^{X}, R^{Y}, R^{Z}, and R^{1A} is independently selected from H, ²H, substituted or unsubstituted alkyl, substituted or unsubstituted alkenyl, substituted or unsubstituted alkynyl, and substituted or unsubstituted heteroalkyl; or R^{X}, R^{Y}, R^{Z}, or R^{1A} together with R^{D} or R^{E} and the atoms to which they are attached form an optionally substituted cycloalkyl (such as a C₃-C₆ cycloalkyl) or an optionally substituted heterocyclic ring (such as a 3- to 15-membered heterocyclic ring);
each of R^{D} and R^{E} is independently selected from H, ²H, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted cycloalkyl (such as a C₃-C₆ cycloalkyl) or a substituted or unsubstituted heterocyclic (such as a 3- to 15-membered heterocyclic ring);
each of R^{F}, R^{G} and R^{H} is independently selected from H, ²H, substituted or unsubstituted alkyl, substituted or unsubstituted alkenyl, substituted or unsubstituted alkynyl, substituted or unsubstituted heteroalkyl, substituted or unsubstituted heterocyclyl, substituted or unsubstituted cycloalkyl, substituted or unsubstituted —C₆-C₁₀ aryl, substituted or unsubstituted 5- to 10-membered heteroaryl, substituted or unsubstituted —CH₂—C₅-C₁₀ aryl, and substituted or unsubstituted —CH₂—C₅-C₁₀ heteroaryl; or alternatively, two or three of R^{F}, R^{G} and R^{H} together with the P⁺ to which they are attached form an optionally substituted heterocyclyl (such as a 3- to 15-membered heterocyclic ring) having, zero, one or more heteroatoms in addition to the P⁺, including, but not limited to, an optionally substituted heteroaryl ring;
or two or three of R^{D}, R^{E}, R^{F}, R^{G} and R^{H} together with the P⁺ to which they are attached form an optionally substituted heterocyclic ring having, zero, one or more heteroatoms in addition to the P⁺, including, but not limited to, a heteroaryl ring; for example, two of R^{E}, R^{F}, and R^{G} are taken together with the P⁺ to form a heterocyclic ring having, zero, one or more heteroatoms in addition to the P⁺.

9. The method of claim 1, wherein the compound has Formula (V): 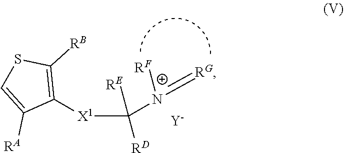
wherein:
Y⁻ is a pharmaceutically acceptable anion;
R^{F} and R^{G} together with the N⁺ to which they are attached form an optionally substituted heteroaryl ring having one or more heteroatoms or an optionally substituted bicyclic heteroaryl ring having one or more heteroatoms;
R^{A} and R^{B} are each independently selected from H, D, halogen, substituted or unsubstituted alkyl, substituted or unsubstituted alkenyl, substituted or unsubstituted alkynyl, nitrile, OR^{I}, NR^{J}R^{K}, NR^{L}C(O)R^{M}, S(O)R^{N}, SO₂R^{O}, SO₂R^{O}R^{P}, SO₂NR^{Q}R^{R}, SO₃R^{S}, CO₂R^{T}, C(O)R^{U}, and C(O)NR^{V}R^{W};
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —NR^{Z}C(O)CR^{X}R^{Y}—, —OC(O)—, —SC(O)—, —NR^{Z}S(O)—, —S(O)NR^{Z}—, —NR^{X}C(O)NR^{Y}—, —C(O)NR^{1A}—, —C(O)O—, —C(O)—, —S(O)—, —S(O)₂—, and —(O)CS—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; or can be taken together with any other R^{X}, R^{Y}, R^{Z}, R^{1A}, R^{D} or R^{E} together with any intervening atoms to form a substituted or unsubstituted ring;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, optionally substituted with halogen, cyclic alkyl, aryl, or heteroaryl, and cycloalkyl; or R^{D} and R^{E} together with the carbon to which they are attached to form a substituted or unsubstituted 3-7-membered cycloalkyl (a C₃-C₇ cycloalkyl) or a substituted or unsubstituted heterocyclic or heteroalkyl ring, or R^{D} and R^{Z} together with the carbon and the —N—C(O)— to which they are attached form an optionally substituted 5-8-membered lactam.

10. The method of claim 1, wherein the compound has Formula (VI) that can be used to treat or prevent pain, itch, and neurogenic inflammation: 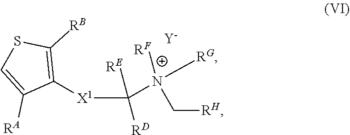
wherein:
Y⁻ is a pharmaceutically acceptable anion;
R^{A} and R^{B} are each independently selected from H, D, halogen, substituted or unsubstituted alkyl, substituted or unsubstituted alkenyl, substituted or unsubstituted alkynyl, OR^{I}, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}, C(O)R^{U}, and C(O)NR^{V}R^{W};
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, and substituted or unsubstituted heteroalkyl;
X¹ is selected from —CR^{X}R^{Y}—, —NR^{Z}C(O)—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; X¹ can also be —NR^{Z}C(O)CR^{X}R^{Y}—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl; or can be taken together with any other R^{X}, R^{Y}, R^{Z}, R^{1A}, R^{D} or R^{E} together with any intervening atoms to form a substituted or unsubstituted ring;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted 3-6-membered cycloalkyl (a C₃-C₆ cycloalkyl), substituted or unsubstituted heterocyclic, or substituted or unsubstituted heteroalkyl ring; or R^{D} and R^{Z} together with the carbon and the —N—C(O)— to which they are attached form an optionally substituted 5-8-membered lactam;
R^{F} and R^{G} together with the N⁺ to which they are attached form an optionally substituted heterocyclic ring having one or more nitrogen atoms; or, each of R^{F} and R^{G} is independently selected from substituted or unsubstituted alkyl, substituted or unsubstituted alkenyl, substituted or unsubstituted alkynyl, substituted or unsubstituted heteroalkyl, and substituted or unsubstituted C₃₋₆ cycloalkyl; and
R^{H} is a substituted or unsubstituted aryl ring, or a substituted or unsubstituted heteroaryl ring.

11. The method of claim 1, wherein the compound has Formula (VII): 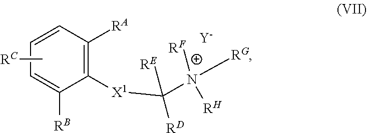
wherein:
Y⁻ is a pharmaceutically acceptable anion;
R^{A} is CO₂R^{T};
R^{T} is substituted or unsubstituted alkyl (preferably methyl or ethyl);
R^{B} is H, D, halogen, or substituted or unsubstituted alkyl; preferably methyl or ethyl;
R^{C} is selected from as H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W}, (preferably H, F, Cl, or CN and more preferably H);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; R^{J} and R^{K} or R^{V} and R^{W} or R^{Q} and R^{R} can also be taken together with the nitrogen to which they are attached to form a substituted or unsubstituted 5, 6, 7, or 8 membered ring;
X¹ can be selected from —CR^{X}R^{Y}—, —NR^{Z}C(O)—, —NR^{Z}C(O)CR^{X}R^{Y}—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; and
each of R^{X}, R^{Y}, R^{Z}, and R^{1A} is independently selected from H, D, substituted or unsubstituted alkyl, substituted or unsubstituted alkenyl, and substituted or unsubstituted alkynyl.

12. The method of claim 1, wherein the compound has Formula (VIII): 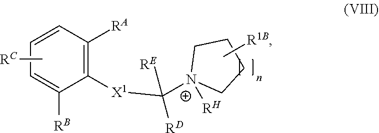
wherein:
Y⁻ is a pharmaceutically acceptable anion;
n is 1, 2, 3, or 4;
R^{A}, R^{B}, and R^{C} are each independently selected from H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W}, (preferably R^{A} and R^{B} are methyl and R^{C} is H, F, Cl, or CN and more preferably H);
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; R^{J} and R^{K} or R^{V} and R^{W} or R^{Q} and R^{R} can also be taken together with the nitrogen to which they are attached to form a substituted or unsubstituted 5, 6, 7, or 8 membered ring;
R^{A}, R^{B}, and/or R^{C} taken together with the phenyl ring to which they are attached can form a fused bicyclic or tricyclic ring system, such as naphthyl, dihydroindenyl, tetrahydronaphthyl, quinolinyl, indolyl, and the like;
X¹ is selected from —CR^{X}R^{Y}—, —NR^{Z}C(O)—, —OC(O)—, —SC(O)—, —C(O)NR^{1A}—, —C(O)O—, —C(O)—, —(O)CS—, —NR^{1A}S(O)—, —S(O)NR^{1A}—, —NR^{1A}C(O)NR^{1A}—, —S(O)— and —S(O)₂—; X¹ can also be —NR^{Z}C(O)CR^{X}R^{Y}—;
each of R^{X}, R^{Y}, R^{Z}, and R^{1A} is independently selected from H, D, substituted or unsubstituted alkyl, substituted or unsubstituted alkenyl, substituted or unsubstituted alkynyl, and substituted or unsubstituted heteroalkyl;
each of R^{D} and R^{E} is independently selected from H, D, substituted or unsubstituted alkyl, substituted or unsubstituted alkenyl, substituted or unsubstituted alkynyl, substituted or unsubstituted heteroalkyl, and substituted or unsubstituted cycloalkyl; or R^{D} and R^{E} together with the carbon to which they are attached form a substituted or unsubstituted 3-6-membered cycloalkyl (a C₃-C₆ cycloalkyl), substituted or unsubstituted heterocyclic, or substituted or unsubstituted heteroalkyl ring;
or R^{D} and R^{Z} together with the carbon and the —N—C(O)— to which they are attached form an optionally substituted 5-8-membered lactam;
R^{H} is a substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; in certain aspects, R^{H} can be a substituted or unsubstituted aralkyl or heteroarylalkyl; in additional aspects, R^{H} can be substituted or unsubstituted alkylcarboxylate, substituted or unsubstituted alkylcarboxamide; and
R^{1B} is a substituted or unsubstituted aryl or substituted or unsubstituted heteroaryl, preferably substituted or unsubstituted phenyl.

13. The method of claim 1, wherein the compound has Formula (IX): 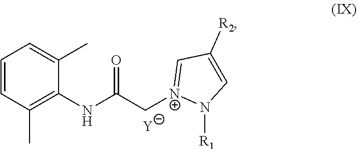
wherein:
Y⁻ is a pharmaceutically acceptable anion;
R¹ is selected from the group consisting of methyl, ethyl, propyl (including n-propyl and isopropyl), butyl (including n-butyl, iso-butyl, tert-butyl, and sec-butyl), cyclohexyl, phenyl, and CH₂C(O)NHR₃;
R² is hydrogen or methyl; and
R³ is hydrogen, methyl, or ethyl.

14. The method of claim 1, wherein the compound has Formula (X): 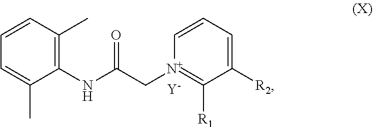
wherein
Y⁻ is a pharmaceutically acceptable anion;
one of R¹ and R² is hydrogen, and the other of R¹ and R² is selected from the group consisting of methyl, ethyl, unsubstituted phenyl, and C(O)OR³; and
R³ is selected from the group consisting of hydrogen, methyl, and ethyl.

15. The method of claim 1, wherein the compound has Formula (XI): 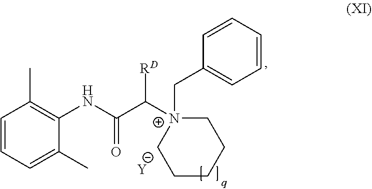
wherein:
Y⁻ is a pharmaceutically acceptable anion;
q is 0, 1, 2, or 3; and
R^{D} is hydrogen, methyl, or ethyl.

16. The method of claim 1, wherein the compound has Formula (XII): 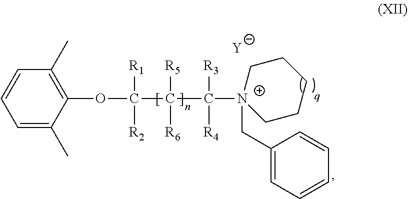
wherein:
Y⁻ is a pharmaceutically acceptable anion;
wherein R₁, R₂, R₃, R₄, R₅, and R₆ are independently selected from hydrogen, C1-C4 alkyl, C1-C4 heteroalkyl, aryl or heteroaryl;
n is 0, 1, 2, 3, 4 and 5;
q is 0, 1, 2, 3, 4 or 5.

17. The method of claim 1, wherein the compound has Formula (XIII): 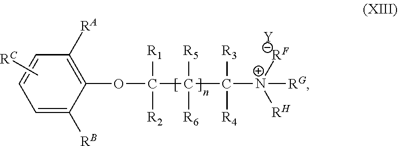
wherein:
Y⁻ is a pharmaceutically acceptable anion;
R^{A}, R^{B}, and R^{C} are each independently selected from H, D, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, OR^{I}, CN, CF₃, NR^{J}R^{K}, NR^{L}C(O)R^{M}, S(O)R^{N}, S(O)₂R^{N}, SO₂R^{O}R^{P}, SO₂NR^{Q}R^{R}, SO₃R^{S}, CO₂R^{T}; C(O)R^{U}, and C(O)NR^{V}R^{W};
each of R^{I}, R^{J}, R^{K}, R^{L}, R^{M}, R^{N}, R^{O}, R^{P}, R^{Q}, R^{R}, R^{S}, R^{T}, R^{U}, R^{V}, and R^{W} is independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; or R^{J} and R^{K} or R^{V} and R^{W} or R^{Q} and R^{R} can also be taken together with the nitrogen to which they are attached to form a substituted or unsubstituted 5, 6, 7, or 8 membered ring;
R^{A}, R^{B}, and/or R^{C} can be taken together with the phenyl ring to which they are attached can form a fused bicyclic or tricyclic ring system, such as naphthyl, dihydroindenyl, tetrahydronaphthyl, quinolinyl, indolyl, and the like;
R₁, R₂, R₃, R₄, R₅, and R₆ are independently selected from hydrogen, C₁-C₄ alkyl, C₁-C₄ heteroalkyl, cycloalkyl, aryl or heteroaryl, preferably hydrogen, methyl or ethyl;
n is 0, 1, 2, 3, 4 and 5;
or R₁, R₂, R₃, R₄, R₅, and/or R₆ together with the carbon(s) to which they are attached form a substituted or unsubstituted cycloalkyl (such as a C₃-C₆ cycloalkyl) or a substituted or unsubstituted heterocyclic (such as a 3- to 15-membered heterocyclic ring);
R^{F} and R^{G} together with the N⁺ form an optionally substituted heterocyclic ring having, zero, one or more heteroatoms in addition to the N⁺, including but not limited to, a heteroaryl ring; for example, two of R^{E}, R^{F}, and R^{G} are taken together with the N⁺ to form a heterocyclic ring having, zero, one or more heteroatoms in addition to the N⁺;
R^{H} is selected from substituted or unsubstituted alkyl, substituted or unsubstituted alkenyl, substituted or unsubstituted alkynyl, such as —CH₂-cycloalkyl, —C₂H₄-cycloalkyl, substituted or unsubstituted —CH₂—C₅-C₁₀ aryl, substituted or unsubstituted —C₂H₄—C₅-C₁₀ aryl, substituted or unsubstituted —CH₂—C₅-C₁₀ heteroaryl, substituted or unsubstituted —C₂H₄—C₅-C₁₀ heteroaryl, —CH₂ OC(O)R^{T}, —CH₂ CO₂R^{T}, —CH₂C(O)NR^{V}R^{W}, —C₂H₄OCOR^{T}, —C₂H₄OR^{I} or
R^{F}, R^{G} and R^{H} together with the N⁺ form substituted or unsubstituted heteroaryl ring (such as pyridinyl or phenyl-pyridinyl) or bridged heterocyclic ring.
